(19) United States
(12) Patent Application Publication
(10) Pub. No.: US 2016/0120828 A1
(43) Pub. Date: May 5, 2016

(54) COLONOSCOPY PREPARATION KIT

(71) Applicant: ASCLEMED USA, INC. dba EnovaChem Manufacturing, Torrance, CA (US)

(72) Inventor: Robert P. Nickell, Torrance, CA (US)

(21) Appl. No.: 14/925,834

(22) Filed: Oct. 28, 2015

Related U.S. Application Data
(60) Provisional application No. 62/072,289, filed on Oct. 29, 2014.

(51) Int. Cl. A61K 31/194; A61K 31/166; A61K 31/07; A61K 31/59; B65D 85/00; A61K 31/4402

(52) U.S. Cl. CPC A61K 31/194; B65D 85/70; A61K 31/4402; A61K 31/07; A61K 31/59; A61K 31/166

(57) ABSTRACT

Provides is a kit comprising: a) a container; and b) following components placed inside of the container: i) magnesium citrate oral Solution; ii) a laxative in form of an oral pharmaceutical dosage form; iii) a topical formulation for application to the skin to relieve irritation; iii) a container of polyethylene glycol powder; iv) an oral pharmaceutical dosage form for treating nausea; and v) an electrolyte drink mix; wherein the kit is used by a patient to clean the patient's colon prior to a colonoscopy.

### CROSS-REFERENCE

The present application claims the benefit of U.S. provisional patent application No. 62/072,289, filed on Oct. 29, 2014, which is incorporated herein by reference in its entirety.

### BACKGROUND SECTION OF THE INVENTION

Colonoscopy is a technique for examining the large intestine for various diseases and conditions. The doctor performing the colonoscopy can see the lining of the large intestine more clearly if the large intestine is free of fecal matter. Therefore, a patient is required to clean his or her large intestine on the day prior to the colonoscopy.

In order to clear the large intestine, the patient has to take laxatives and other medications the day before the colonoscopy. The patient typically receives an instruction sheet from a doctor as to the medications the patient has to purchase and how to use them. Multiple problems arise with having a patient in charge of purchasing the various medications. First, the patient, who may have limited education, may purchase the wrong medication. Second, once the colonoscopy preparation starts, the patient has a constant diarrhea. The patient has to stay close to a toilet at all times. If the patient forgets or makes a mistake in purchasing all of the necessary medications, the patient's diarrhea may not allow the patient to leave his or her house and go back to a pharmacy.

There is a need in the prior art for a colonoscopy kit that addresses the above problems.

### SUMMARY SECTION OF THE INVENTION

Provided is a kit comprising a container and following components placed inside the container: a) magnesium citrate oral Solution, b) a laxative in form of an oral pharmaceutical dosage form, c) a topical formulation, such as in the form of an ointment, for application to the skin to relieve irritation; d) polyethylene glycol powder, e) metoclopramide HCl tablet; and f) an electrolyte drink mix. The kit can further comprise a container of water. The kit can further comprise a beverage such as a sport drink (Gatorade or Powerade). The water and/or beverage can be used for mixing of the powder (s). Each of these components can be placed in its own package or container.

Provided is a kit comprising: a) a container, and b) following components placed inside of the container: i) magnesium citrate oral Solution; ii) a laxative in form of an oral pharmaceutical dosage form; iii) a topical formulation for application to the skin to relieve irritation; iii) a container of polyethylene glycol powder, iv) an oral pharmaceutical dosage form for treating nausea; and v) an electrolyte drink mix, wherein the kit is used by a patient to clean the patient's colon prior to a colonoscopy. The kit can further comprise a container of water or a beverage for mixing with the polyethylene glycol powder and the electrolyte drink mix. The kit can comprise one or two bottles of magnesium citrate oral solution. The concentration of the magnesium citrate oral solution in the bottle can be 1.745 g/fl oz. The laxative in the form of the oral pharmaceutical dosage form can be bisacodyl. The laxative can be 1 to 6 bisacodyl enteric coated tablets at a dosage of 5 mg. The topical formulation can be an ointment. The ointment can be a vitamins A & D ointment. The ointment can be placed in a package. The oral dosage form for treating nausea can be metoclopramide HCl tablet. The kit can further comprise one or more gloves. Each component of the kit can be placed in its own separate container or package. The kit can further comprise an instruction sheet. The instruction sheet can state to make a mixture in a liquid with the polyethylene glycol powder and the oral electrolyte drink Mix. The container inside of which the components of the kit are placed can be a box with a lid. Provides is a kit comprising: a) a container, and b) following components placed inside of the container: i) 1-2 bottles of magnesium citrate oral solution; ii) 1-6 bisacodyl enteric coated tablets at a dosage of 5 mg each; iii) 1-5 vitamins A & D ointment packages; iv) 1 bottle of polyethylene glycol powder, v) 1-2 tablets of 5-10 mg metoclopramide HCl; and vi) 1-2 packages of rice-based oral electrolyte drink mix; wherein the kit is to be used by a patient to clean the patient's colon prior to a colonoscopy; and wherein each of the components of the kit is placed in its own package or container. Provided is a kit comprising a) a container, and b) following components placed inside of the container: i) 1 bottle of magnesium citrate oral solution; ii) 4 bisacodyl enteric coated tablets at a dosage of 5 mg each; iii) 3 vitamins A & D ointment packages; iv) 1 bottle of polyethylene glycol powder; v) 2 tablets of 10 mg metoclopramide HCl; and vi) 2 packages of rice-based oral electrolyte drink Mix; wherein the kit is to be used by a patient to clean the patient's colon prior to a colonoscopy; and wherein each of the components of the kit is placed in its own package or container. The kit can further comprise a pair of gloves.

### BRIEF DESCRIPTION OF THE FIGURES

FIG. 1 illustrates a blister pack with tablets in each blister.

FIG. 2 illustrates a box for holding a blister pack of tablets.

FIG. 3 illustrates a bottle holding liquid therein.

FIG. 4 illustrates packets of ointments, and a box for holding them.

FIG. 5 illustrates a packet for holding a electrolyte mix powder.

FIG. 6 illustrates a bottle for holding polyethylene glycol powder.

FIG. 7 illustrates a glove.

FIG. 8 illustrates an instruction sheet.

FIG. 9 illustrates a box with a lid for placement of the different components of the kit.

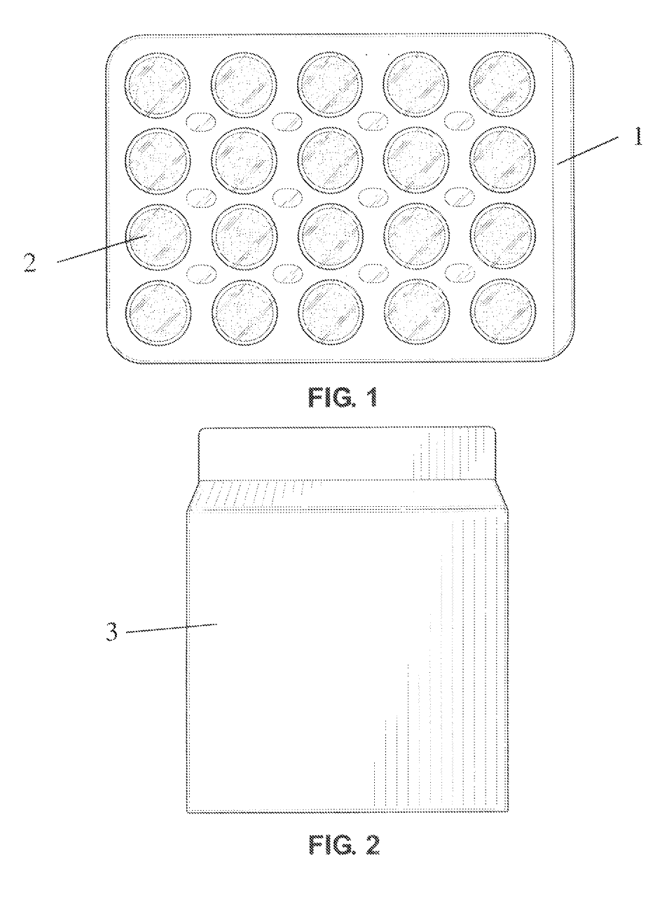

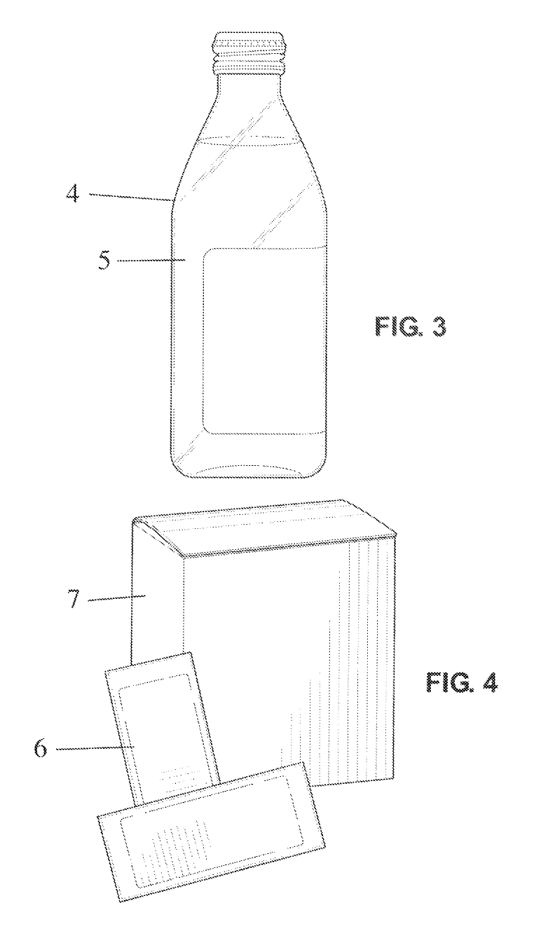

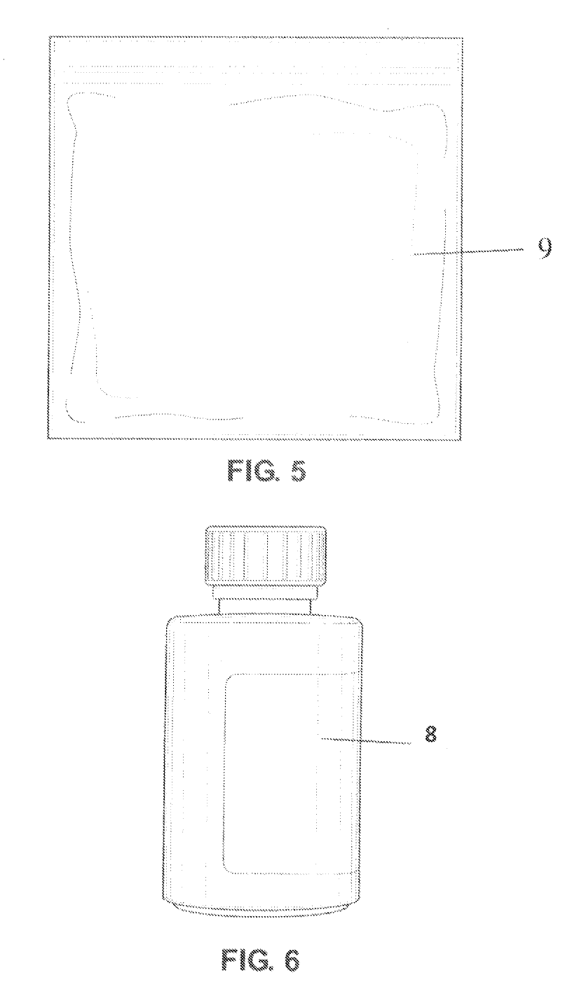

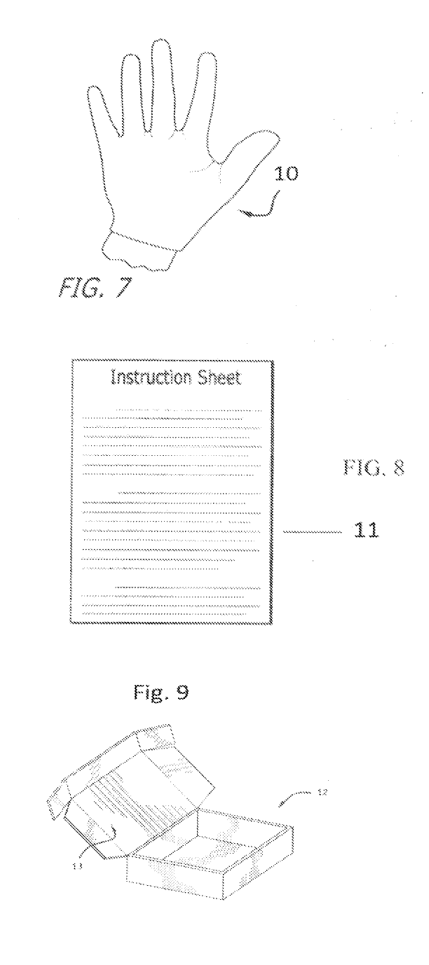

### DETAILED DESCRIPTION OF THE INVENTION

The present invention provides a kit with all the items that a patient needs to prepare a colonoscopy. The patient no longer needs to decide which items, such as particular medication, to purchase. The kit minimizes patient error in purchasing the wrong medication or forgetting a necessary item.

The components of the kit can be put in a container, such as a box 12. The box 12 can be a rectangular cube which has a lid 13 on top for opening the box and accessing the components of the kit.

One component of the kit can be magnesium citrate oral solution 5, which is a saline laxative. One or two containers, typically bottles 4, of the magnesium citrate solution 5 can be placed in the kit. The concentration of the solution can be about 1.5 to about 2 g/per fl oz. The solution can be flavored, such as lemon, cherry, or grape flavored. The bottle 4 can contain 1.745 g of magnesium citrate, and have a volume of 10 fl oz (296 ml).

One component of the kit can be a laxative in the form of an oral pharmaceutical dosage form, typically a tablet 2. The laxative can be bisacodyl. The kit can contain 1-6 bisacodyl enteric coated tablets, such as four tablets, typically in a dosage of 5 mg. The tablets 2 can be provided in a blister 1 or non-blister packet.

The kit can comprise a topical formulation, such as in the form of an ointment or a cream, for application to the skin to relieve irritation. In one embodiment, 1-5 vitamins A & D ointment packets 6 can be included in the kit, with or without an accompanying box 7 for holding the packets 6.

The kit can comprise a container of polyethylene glycol powder 8, which is an osmotic laxative. The Polyethylene glycol can be polyethylene glycol 3350. The polyethylene glycol powder can be sugar free and/or with flavor, and can be in a bottle 8.

The kit can comprise oral pharmaceutical dosage form to treat nausea and vomiting, such as metoclopramide HCl, typically in the form of a tablet 2. About 1-2 tablets of 5 mg to 10 mg metoclopramide HCl tablet can be included in the kit. The tablets can be provided in a blister 1 or a non-blister packet.

The kit can comprise an electrolyte drink mix. The electrolyte drink mix can be a Rice-based oral electrolyte drink mix, such as CeraLyte® 50, to prevent dehydration. The electrolyte drink mix can be in its own packet 9.

The kit can contain an instruction sheet 11. The instruction sheet 11 can identify one or more components of the kit, provide instructions as to how to use each component, and optionally suggest an order of use for the components of the kit.

The kit can further comprise a bottle 4 of water 5 (numeral 5 denotes liquid inside of bottle 4), or a beverage such as a sport drink (Gatorade® or Powerade®). The water and/or beverage can be used for mixing of the polyethylene glycol powder and the Rice-based oral powder.

The kit can further include one or a pair of gloves 10. The gloves 10 can be a pair of nitrile powder free sterile gloves, and may come in its own packaging.

The instruction sheet 11 can instruct the patient to take a number of bisacodyl enteric coated 5 mg tabs at about 1 pm. The patient is also instructed to mix the GaviLAX® (polyethylene glycol powder) with 64 ounces of water (or other beverage) and add 2 packs of CeraLyte® powder (Rice Based Oral Electrolyte), mix well, refrigerate, and starting around 5 pm, drink 8 oz. approximately every 10 minutes until completed. The patient can be instructed to take a number of bisacodyl enteric coated 5 mg tablets (or other dosages) at around 8 pm, and a number of metoclopramide 10 mg tablets (or other dosages) after 5 pm. The patient can be instructed to drink the Magnesium citrate oral solution after 5 pm. The patient can be instructed to use vitamin A&D ointment as needed to help soothe anal tissue.

The following tables provide for specific examples of kits:

The following tables provide examples of the components of the kit.

TABLE 1
1-2 bottles Magnesium Citrate Oral Solution (1.745 g/fl oz)
(Lemon Flavored)
1-6 Bisacodyl Enteric Coated Tablets 5 mg or other type of laxative
1-5 Vitamins A & D Ointment Packets
1 bottle Polyethylene Glycol 3350. Sugar Free and/or with flavor
1-2 tabs of 5-10 mg metoclopramide HCl tablet
1-2 packs CeraLyte ® 50 Rice-based Oral Electrolyte Drink Mix sugar
free, or not or flavored
1 pair of nitrile powder free sterile gloves (size 7.5)

TABLE 2
1-2 bottles Magnesium Citrate Oral Solution (1.745 g/fl oz)
(Lemon Flavored)
1-6 Bisacodyl Enteric Coated Tablets 5 mg or other type of laxative
1-5 Vitamins A & D Ointment Packets
1 bottle Polyethylene Glycol 3350. Sugar Free and/or with flavor
1-2 tabs of 5-10 mg metoclopramide HCl tablet
1-2 packs CeraLyte ® 50 Rice-based Oral Electrolyte Drink Mix sugar
free, or not or flavored

TABLE 3
1 bottles Magnesium Citrate Oral Solution (1.745 g/fl oz)
(Lemon Flavored) (296 ml) (Distributed by Major Pharmaceuticals)
4 Bisacodyl Enteric Coated Tablets 5 mg (Distributed by Major
Pharmaceuticals)
3 Vitamins A & D Ointment Packets 5 g
Contains White petroleum, USP, 75% w/w, lanolin, light liquid paraffin,
vitamin A. and Vitamin D. (Dynarex Corporation)
1 bottle GaviLax Polyethylene Glycol 3350. Sugar Free (Gavis
Pharmaceuticals)
2 tablets of 10 mg metoclopramide HCl tablet, USP (Mylan Institutional,
Inc.)
2 packs CeraLyte ® 50 Rice-based Oral Electrolyte Drink Mix sugar free,
or not or flavored. CeraLyte ® 50 provides (per liter, reconstituted),
sodium 50 mEq, potassium 20 mEq, citrate 30 mEq, chloride 40 mEq;
mOsm <200; carbohydrates 40 g
1 pair of nitrile powder free sterile gloves (size 7.5)

What is claimed is:

1. A kit comprising:
a) a container; and
b) following components placed inside of the container: i) magnesium citrate oral Solution; ii) a laxative in form of an oral pharmaceutical dosage form; iii) a topical formulation for application to the skin to relieve irritation; iii) a container of polyethylene glycol powder; iv) an oral pharmaceutical dosage form for treating nausea; and v) an electrolyte drink mix; wherein the kit is used by a patient to clean the patient's colon prior to a colonoscopy.

2. The kit of claim 1, wherein the kit further comprises a container of water or a beverage for mixing with the polyethylene glycol powder and the electrolyte drink mix.

3. The kit of claim 1, wherein the kit comprises one or two bottles of Magnesium Citrate Oral Solution.

4. The kit of claim 3, wherein a concentration of the magnesium citrate oral solution in the bottle is 1.745 g/oz.

5. The kit of claim 1, wherein the laxative in the form of the oral pharmaceutical dosage form is Bisacodyl.

6. The kit of claim 5, wherein the laxative is 1 to 6 bisacodyl enteric coated tablets at a dosage of 5 mg.

7. The kit of claim 1, wherein the topical formulation is an ointment.

8. The kit of claim 7, wherein the ointment is a vitamins A & D ointment.

9. The kit of claim 8, wherein the ointment is placed in a package.

10. The kit of claim 1, wherein the oral dosage form for treating nausea is metoclopramide HCl tablet.

11. The kit of claim 1, further comprising one or more gloves.

12. The kit of claim 1, wherein each component of the kit is placed in its own separate container or package.

13. The kit of claim 1, further comprising an instruction sheet.

14. The kit of claim 13, wherein the instruction sheet states to make a mixture in a liquid with the polyethylene glycol powder and the oral electrolyte drink mix

15. The kit of claim 1, wherein the container inside of which the components are placed is a box with a lid.

16. A kit comprising
a) a container; and
b) following components placed inside of the container:
i) 1 bottle of magnesium citrate oral solution;
ii) 4 bisacodyl enteric coated tablets at a dosage of 5 mg each;
iii) 3 vitamins A & D ointment packages;
iv) 1 bottle of polyethylene glycol powder;
v) 2 tablets of 10 mg metoclopramide HCl; and
vi) 2 packages of rice-based oral electrolyte drink Mix;
wherein the kit is to be used by a patient to clean the patient's colon prior to a colonoscopy; and
wherein each of the components of the kit is placed in its own package or container.

17. The kit of claim 16, further comprising a pair of gloves.
